(19) United States
(12) Patent Application Publication
(10) Pub. No.: US 2026/0069834 A1
(43) Pub. Date: Mar. 12, 2026

(54) Lock Solution Having Magnetic Nanoparticles

(71) Applicant: Becton, Dickinson and Company, Franklin Lakes, NJ (US)

(72) Inventors: Ajay Suryavanshi, Pune (IN); Shishir Prasad, Ramsey, NJ (US); Jack Balji, Mahwah, NJ (US); Chetan Satish Dhuri, Bangalore (IN)

(21) Appl. No.: 18/883,588

(22) Filed: Sep. 12, 2024

(51) Int. Cl. A61M 25/06

(52) U.S. Cl. CPC A61M 25/0606; A61M 2205/0205; A61M 2205/0244; A61M 2205/0288; A61M 2205/3306; A61M 2205/3368; A61M 2205/502; A61M 2205/583; A61M 2230/50

(57) ABSTRACT

Provided herein is a system including an IV catheter assembly with a catheter having a distal end positionable intravenously within a patient and a port providing fluid access to the catheter. The system also includes a pre-filled syringe connectable to the port and that contains an aqueous catheter lock solution having magnetic particles suspended therein. The system further includes a probe module comprising a magnetic probe that includes a magnet element and a driver. The magnetic probe is operable in a first driving mode where the magnet element causes the magnetic nanoparticles to migrate towards the distal end of the catheter and is operable in a second driving mode where the magnet element causes a temperature of the magnetic nanoparticles to increase, generating a magnetically-induced hyperthermic condition at the distal end of the catheter that prevents and/or treats thrombus formation and biofilm accumulation in the catheter.

### BACKGROUND OF THE INVENTION

### Field of the Invention

The present disclosure relates generally to intravenous (IV) catheters and, more specifically, to devices for use with IV catheters that prevent and/or treat the adherence of blood clots and/or biofilms within the catheter.

### Description of Related Art

Vascular access devices (VADs) are used in the medical field to access peripheral and/or central vasculature of a patient for purposes of infusion therapy and/or blood withdrawal. Common types of VADs include over-the-needle peripheral intravenous catheters (PIVCs), peripherally inserted central catheters (PICCs), central venous catheters (CVCs), midline catheters, and other tubular systems (e.g., urinary catheters, stents, etc.). The VAD may be indwelling for short term (days), moderate term (weeks), or long term (months to years).

Some of the most common complications associated with indwelling intravenous (IV) catheters are the issues of catheter occlusion/blockage and the risk of contamination of the IV catheter that may lead to transmitting of a catheter related bloodstream infection (CRBSI) to a patient.

With regard to the occlusion of the IV catheter, it is recognized that blood clots or other thrombotic occlusions may attach to the inner wall or surface of the IV catheter, either along a length of the catheter or at the tip of the catheter. As a result of such catheter occlusions, the IV catheter may need to be removed from the patient, with it being recognized that such catheter removal is time consuming, uncomfortable for the patient, and expensive due to increased length of stay and therapy interruptions.

With regard to contamination of the IV catheter, it is common for microbial biofilms to develop on the inner wall or surface of the IV catheter. In the development of such biofilms, bacteria may aggregate and proliferate on the inner wall or surface of the IV catheter, with the bacteria excreting an encasing exopolysaccharide ‘slime’ that consolidates their attachment to IV catheter wall and the microaggregates differentiating into characteristic biofilms.

Several methods exist for preventing the formation of blood clots and biofilms within an IV catheter. As a first example, regular flushing of the IV catheter with saline or heparin may be employed to prevent the build-up of blood clots and bacterial microorganisms that can lead to biofilm formation. However, such flushing of the IV catheter is labor intensive for healthcare providers (as they must continuously monitor the saline levels and reinstall new saline bags when required), and the patient must be connected to the line at all times. As a second example, antimicrobial catheters may be used that are designed to release agents that prevent the growth of bacteria and other microorganisms, which can help to prevent biofilm formation. However, the antimicrobial effects of catheters may wear off with time. As a third example, antibiotic lock therapy may be used with the IV catheter, which involves instilling an antibiotic solution into the IV catheter lumen and leaving it in place for a specific duration in order to prevent the growth of bacteria and other microorganisms. However, overexposure to antibiotics may lead to an increase in antibiotic resistant microorganisms, i.e., antimicrobial resistance (AMR). As a fourth example, a device (e.g., including a piezoelectric actuator) may be employed to introduce oscillations or vibrations into a standing column of fluid contained or locked within the IV catheter, with the oscillations or vibrations introduced into the fluid acting to prevent attachment of occlusions and/or a biofilm within the indwelling catheter. However, such vibration inducing devices may not be effective with longer IV catheters (e.g., PICCs) and/or may not adequately address occlusion of the IV catheter at the distal tip thereof, where occlusions are most prevalent.

Accordingly, a need exists in the art for an apparatus that provides for treatment/prevention of occlusions and biofilms within an indwelling IV catheter.

### SUMMARY OF THE INVENTION

Provided herein is a system including an IV catheter assembly with a catheter having a distal end positionable intravenously within a patient and a port providing fluid access to the catheter, wherein a lumen of the catheter may retain a fluid therein. The system also includes a pre-filled syringe connectable to the port, the pre-filled syringe comprising a syringe barrel and a plunger assembly movable within the syringe barrel, with the syringe barrel defining a chamber that contains an aqueous catheter lock solution having magnetic particles suspended therein. The system further includes a probe module comprising a magnetic probe that includes a magnet element and a driver, the magnetic probe positionable adjacent the IV catheter assembly and to cause movement and heating of the magnetic particles in the aqueous catheter lock solution. The magnetic probe is configured to operate in a first driving mode where the magnet element causes the magnetic nanoparticles to migrate towards the distal end of the catheter and operate in a second driving mode where the magnet element causes a temperature of the magnetic nanoparticles to increase, generating a magnetically-induced hyperthermic condition at the distal end of the catheter, wherein the magnetically-induced hyperthermic condition at the distal end of the catheter prevent and/or treats thrombus formation and biofilm accumulation in the catheter.

In some embodiments, the magnetic particles comprise iron oxide nanoparticles.

In some embodiments, the magnetic particles are loaded with an antithrombotic or antimicrobial material.

In some embodiments, the magnet element comprises a magnetic coil or a plurality of magnets.

In some embodiments, when operating in the second driving mode, the magnetic probe is configured to generate an alternating magnetic field that causes a temperature of the magnetic particles to increase.

In some embodiments, the alternating magnetic field activates the magnetic particles via magnetic coupling between a magnetic component of the magnetic field and a magnetic moment of the magnetic particles, with the magnetic particles absorbing the energy from this coupling and dissipating it as heat, to generate the hyperthermic condition.

In some embodiments, when operating in the second driving mode, the magnetic probe is configured to heat the aqueous catheter lock solution to 40-70 degrees Celsius.

In some embodiments, the magnetic probe comprises a temperature sensor to measure skin temperature at a location adjacent the distal end of the catheter.

In some embodiments, the magnetic particles are coated with up-conversion nanoparticles that shift a florescence signal of the magnetic particles into near infrared.

In some embodiments, the magnetic probe further comprises an infrared or near infrared light source configured to charge the up-conversion nanoparticles and an optical sensor configured to detect the florescence signal of the magnetic particles after charging thereof, so as to enable location tracking of the magnetic particles within the catheter.

In some embodiments, the probe module further comprises a display configured to display settings of the magnetic probe and/or a value of one or more parameters measured by the magnetic probe and a user interface configured to input settings for the magnetic probe, wherein the display and the user interface are provided on the probe or on a docking station or electronic control unit associated with the magnetic probe.

In some embodiments, the magnetic probe has an indicator light configured to indicate whether the magnetic probe is operating in the first driving mode or the second driving mode.

Also provided herein is a method for preventing and/or treating thrombus formation and biofilm accumulation in an indwelling IV catheter. The method includes coupling a pre-filled syringe to a port of an intravenous (IV) catheter assembly including a catheter having a distal end positioned intravenously within a patient, wherein the port provides fluid access to the catheter. The method also includes injecting an aqueous catheter lock solution into the catheter via the pre-filled syringe, the aqueous catheter lock solution having magnetic particles suspended therein. The method further includes operating a magnetic probe to cause movement and heating of the magnetic particles in the aqueous catheter lock solution injected into the catheter, wherein operating the magnetic probe comprises operating the magnetic probe in a first driving mode to cause the magnetic nanoparticles to migrate towards the distal end of the catheter and operating the magnetic probe in a second driving mode to cause a temperature of the magnetic nanoparticles to increase, generating a magnetically-induced hyperthermic condition at the distal end of the catheter, wherein the magnetically-induced hyperthermic condition at the distal end of the catheter prevents and/or treats thrombus formation and biofilm accumulation in the catheter.

In some embodiments, operating the magnetic probe in the second driving mode comprises generate an alternating magnetic field that causes a temperature of the magnetic particles to increase.

In some embodiments, the method further includes measuring skin temperature at a location adjacent the distal end of the catheter magnetic probe via a temperature sensor included in the magnetic probe.

In some embodiments, the magnetic particles are coated with up-conversion nanoparticles that shift a florescence signal of the magnetic particles into near infrared, and the method further comprises emitting infrared or near infrared light from a light source toward the magnetic particles, in order to charge the up-conversion nanoparticles and detecting the florescence signal of the magnetic particles after charging thereof via an optical sensor, so as to enable location tracking of the magnetic particles within the catheter.

In some embodiments, operating the magnetic probe in the second driving mode comprises pre-programming the magnetic probe to run for a pre-set period of time required to achieve a target temperature for the aqueous catheter lock solution that creates the magnetically-induced hyperthermic condition at the distal end of the catheter.

In some embodiments, the target temperature of the aqueous catheter lock solution is 40-70 degrees Celsius.

In some embodiments, the magnetic particles are loaded with an antithrombotic or antimicrobial material.

In some embodiments, the method further includes retrieving the aqueous catheter lock solution out from the catheter after generating the magnetically-induced hyperthermic condition at the distal end of the catheter.

Also provided herein is a system including a tubular structure having a distal end positionable intravenously within a patient and a lumen configured to retain a fluid therein. The system also includes a pre-filled syringe that may be placed in fluid communication with the tubular structure, the pre-filled syringe comprising a syringe barrel and a plunger assembly movable within the syringe barrel, with the syringe barrel defining a chamber that contains an aqueous catheter lock solution having magnetic particles suspended therein. The system further includes a probe module comprising a magnetic probe that includes a magnet element and a driver, the magnetic probe positionable in proximity to the tubular structure to cause movement and heating of the magnetic particles in the aqueous catheter lock solution. The magnetic probe is configured to operate in a first driving mode where the magnet element causes the magnetic nanoparticles to migrate towards the distal end of the tubular structure and operate in a second driving mode where the magnet element causes a temperature of the magnetic nanoparticles to increase, generating a magnetically-induced hyperthermic condition at the distal end of the tubular structure, wherein the magnetically-induced hyperthermic condition at the distal end of the tubular structure prevents and/or treats thrombus formation and biofilm accumulation in the tubular structure.

### BRIEF DESCRIPTION OF THE DRAWINGS

FIG. 1 is a perspective view of an IV catheter assembly, with which embodiments of the present disclosure may be employed;

FIG. 2 is a perspective view of a system for prevention and/or treatment of catheter occlusions and biofilms within the IV catheter assembly of FIG. 1, according to an aspect of the present disclosure;

FIG. 3A shows a perspective view of a magnetic probe module included in the system of FIG. 2, according to an aspect of the present disclosure;

FIG. 3B is a block schematic diagram of the magnetic probe module of FIG. 3A;

FIG. 4A shows a perspective view of a magnetic probe module included in the system of FIG. 2, according to another aspect of the present disclosure;

FIG. 4B is a block schematic diagram of the magnetic probe module of FIG. 4A;

FIG. 5 is a block schematic diagram of a magnetic probe module included in the system of FIG. 2, according to another aspect of the present disclosure;

FIG. 6 is a block schematic diagram of a magnetic probe module included in the system of FIG. 2, according to another aspect of the present disclosure;

FIG. 7 is a flowchart illustrating a method for preventing and/or treating thrombus formation and biofilm accumulation in an indwelling IV catheter, according to an aspect of the present disclosure;

FIG. 8 is a graphical illustration of certain steps of the method of FIG. 7; and

FIG. 9 is a graphical illustration of magnetic nanoparticle-induced hyperthermia preventing and/or treating thrombus formation and biofilm accumulation at the distal end of in an indwelling IV catheter.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, equivalents, variations, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

As used in this specification, the words “proximal” and “distal” refer to the direction closer to and away from, respectively, a user who would place the device into contact with a patient. Thus, for example, the end of a device first touching the body of the patient would be the distal end, while the opposite end of the device being manipulated by the user would be the proximal end of the device.

The terms “first”, “second”, and the like are not intended to refer to any particular order or chronology, but refer to different conditions, properties, or elements.

As used herein, “at least one of” is synonymous with “one or more of.” For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

Provided herein are systems and methods for maintaining patency of an indwelling IV catheter, such as a peripherally inserted central catheter (PICC) or central venous catheter (CVC) as non-limiting examples, with the systems/methods operating to introduce a catheter lock solution having magnetic particles suspended therein into the IV catheter and generate a hyperthermic condition at the distal end of the IV catheter via the catheter lock solution, so as to prevent /d/ or treat blood clots/biofilms within the catheter. While certain vascular access devices (VADs) or catheter assemblies are discussed below and exemplified in the attached drawings with which such devices/methods may be implemented, those of skill will appreciate that any number of different VADs and/or catheter assemblies may be used within the scope of the present disclosure.

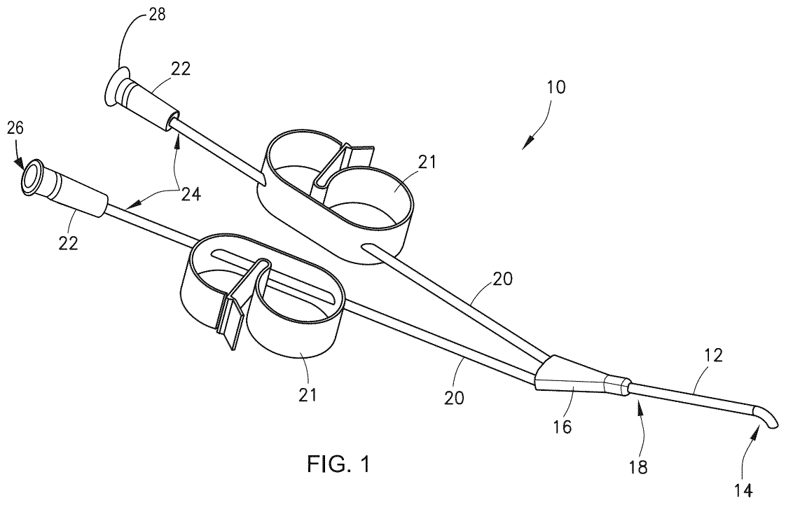

Reference is first made to FIG. 1, which depicts an intravenous (IV) catheter assembly 10 with which aspects of the disclosure may be implemented, according to a non-limiting embodiment. The IV catheter assembly 10 includes a catheter 12 having a distal end 14 that may be inserted transcutaneously through the skin of a patient at an insertion site. The IV catheter assembly 10 also includes a hub 16 coupled to a proximal end 18 of the catheter 12. In the illustrated embodiment, the hub 16 is configured as a bifurcation hub 16, with the catheter assembly 10 further including a plurality of extension legs 20 that operably connect, via the bifurcation hub 16, to a corresponding number of lumens defined by the catheter 12. In some embodiments, a clamp 21 may be provided on each extension leg 20 to control a flow of fluid therethrough by selectively sealing off the extension leg 20.

Each of the extension legs 20 includes a luer hub 22 positioned at a proximal end 24 thereof, with the luer hub providing a (input) port to the IV catheter assembly 10. As used herein, a “luer” hub refers to a connector that includes a tapered portion (i.e., a luer taper) for creating a friction engagement between a tapered stem or elongated member of a male luer connector and a tapered cavity. Each luer hub 22 is configured as a female luer connection having a tapered cavity 26 configured to receive and engage a tapered stem or elongated member of a male luer connector, as well as a threaded outer surface 28 configured to engage threads on the inner surface of an annular shield of the male luer connector.

While hub 16 is shown in FIG. 1 as a bifurcation hub 16, in other embodiments, the hub may be a singular hub 16 having a luer hub 22 formed directly thereon at its proximal end. As described above, such a luer hub 22 is configured as a female luer connection having a tapered cavity 26 configured to receive and engage a tapered stem or elongated member of a male luer connector.

In still other embodiments, the IV catheter assembly 10 may include a catheter adapter (e.g., a y-adapter) rather than a hub 16. Such a catheter adapter may include one or more extension legs extending proximally therefrom, with such extension legs 20 including a luer hub 22 at the proximal end thereof, as previously described.

It is recognized that embodiments of the disclosure, as described here below, may be incorporated in and/or used with any of the catheter assemblies as described above, or with still other catheter assemblies not described herein. Thus, it is understood that aspects of the disclosure are not meant to be limited to use only with the specific catheter assemblies described here below.

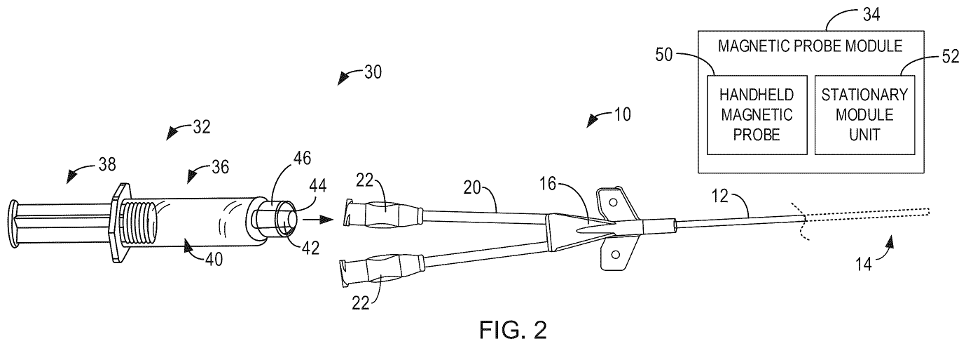

Referring now to FIG. 2, shown is a non-limiting embodiment of a system 30 that includes the catheter assembly 10 of FIG. 1, along with a pre-filled syringe 32 containing a magnetic nanoparticle-laden catheter lock solution, and a magnetic probe module 34 operable to maneuver and locally heat up the magnetic nanoparticles when the lock solution is injected/locked in the indwelling catheter 12, thereby generating a hyperthermic condition at the distal end 14 of the catheter 12 that treats and/or prevents the formation of occlusions (i.e., thrombus) and/or biofilms at the distal end 14 of the catheter 12.

As shown in FIG. 2, the pre-filled syringe 32 may be coupled to the luer hub 22 of one of extension legs 20. The syringe 32 generally includes a syringe barrel 36 and a plunger assembly 38, with a chamber 40 defined by the barrel and proximal of the plunger assembly that may retain fluid therein. The plunger assembly 38 is axially movable within the syringe barrel 36 to an advanced position to facilitate administering of an injectable fluid to a patient.

A distal end of the syringe barrel 36 includes a tip 42 that extends distally outward and defines a lumen 44 in fluid communication with the chamber 40. A luer lock adapter 46 is mounted on an external surface of the tip 42 that is couplable to a luer hub 22 of catheter assembly 10, to enable a transfer of fluid into the catheter assembly 10.

As previously described, the syringe 32 may be provided as a pre-filled syringe that contains a fluid within chamber 40. The fluid may be in the form of a saline solution or any other isotonic liquid that includes magnetic particles suspended therein-with the magnetic particles allowing for a hyperthermic condition to be generated at the distal end 14 of the IV catheter that acts to treat and/or prevent the formation of occlusions (i.e., thrombus) and/or biofilms at the distal end 14 of the catheter 12. Magnetic nanoparticles are suitably coated to avoid aggregation and loss of hyperthermia efficiency. The magnetic particle-laden catheter lock solution may be injected into the catheter assembly 10 via the syringe 32, with it recognized that blood retained in the catheter 12 may also mix with the catheter lock solution to provide an overall standing column of fluid within the catheter assembly 10. The catheter lock solution may be introduced into the catheter assembly 10 following a blood draw procedure or injection of a medicament into a patient, with the catheter lock solution flushing the luer hub 22 and being received within a fluid lumen in the catheter 12.

According to embodiments, the magnetic particles may comprise magnetic nanoparticles (or “nanobots”). The magnetic nanoparticles may be composed of iron oxide or iron-oxides phases (i.e., Fe₃O₄ and Fe₂O₃) configured as nanorods. The magnetic nanoparticles may also be composed of other magnetic ferrites in spinel structures doped with Mn, Co, Ni or Zn. The magnetic nanoparticles may also be coated with both organic and inorganic compounds to improve the biocompatibility and target specificity thereof, as well as prevent aggregation of the nanoparticles. As explained in further detail below, the magnetic nanoparticles are configured to respond to external magnetic fields applied thereto, to provide for remote manipulation of the magnetic nanoparticles, such as moving the magnetic nanoparticles to a desired location and heating the magnetic nanoparticles.

In some embodiments, the catheter lock solution in syringe 32 may also include antithrombotic or antimicrobial material therein. That is, the magnetic nanoparticles in the catheter lock solution may be loaded with an antithrombotic and/or antimicrobial material that enables prevention and/or treatment of thrombotic occlusions and/or biofilms and microbes that may be present at the distal end/tip 14 of the catheter 12. In some embodiments, the antithrombotic may comprise alteplase or another thrombolytic medication, while the antimicrobial may comprise cefazolin, ceftazidime, gentamicin, linezolid, vancomycin, or the like. The antithrombotic and/or antimicrobial material may bind to the magnetic nanoparticles and may be delivered to a target location (e.g., the distal end 14 of the catheter 12) along with the magnetic nanoparticles.

In some embodiments, the magnetic nanoparticles may also be coated with up-conversion nanoparticles that shift a florescence signal of the magnetic nanoparticles into near infrared when the up-conversion nanoparticles are charged by an infrared or near infrared light source. The florescence signal (i.e., infrared signal) generated by the up-conversion nanoparticles after charging thereof enables location tracking of the magnetic particles within the catheter by the magnetic probe module 34, as explained in further detail below.

As shown in FIG. 2, the magnetic probe module 34 includes a handheld magnetic probe 50 and a stationary module unit 52 that, according to embodiments, may comprise a docking station or control panel, as explained in further detail below. The magnetic probe 50 may be positioned adjacent the catheter assembly 10 and, in particular, may be positioned proximate to an insertion site of the catheter 12 that has been inserted intravenously into the patient. That is, the magnetic probe 50 may be grasped and manipulated by a user to provide for positioning thereof at a desired location. As explained in further detail below, the magnetic probe 50 includes one or more magnetic elements and an associated driver that are operable to cause movement and heating of the magnetic particles in the catheter lock solution-with the magnetic probe 50 operable in a first driving mode that causes the magnetic nanoparticles to migrate towards the distal end 14 of the catheter 12 and operable in a second driving mode that causes a temperature of the magnetic nanoparticles to increase, thereby generating a magnetically-induced hyperthermic condition at the distal end 14 of the catheter 12 (i.e., in a distal portion of the standing column of catheter lock fluid contained within the catheter 12) that prevents and/or treats thrombus formation and biofilm accumulation in the catheter 12.

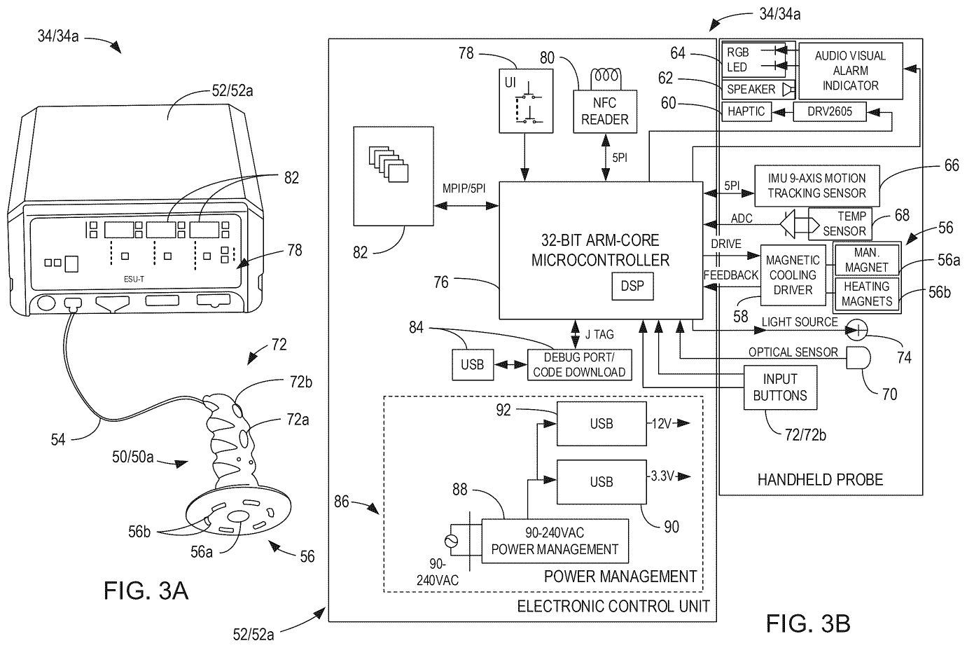

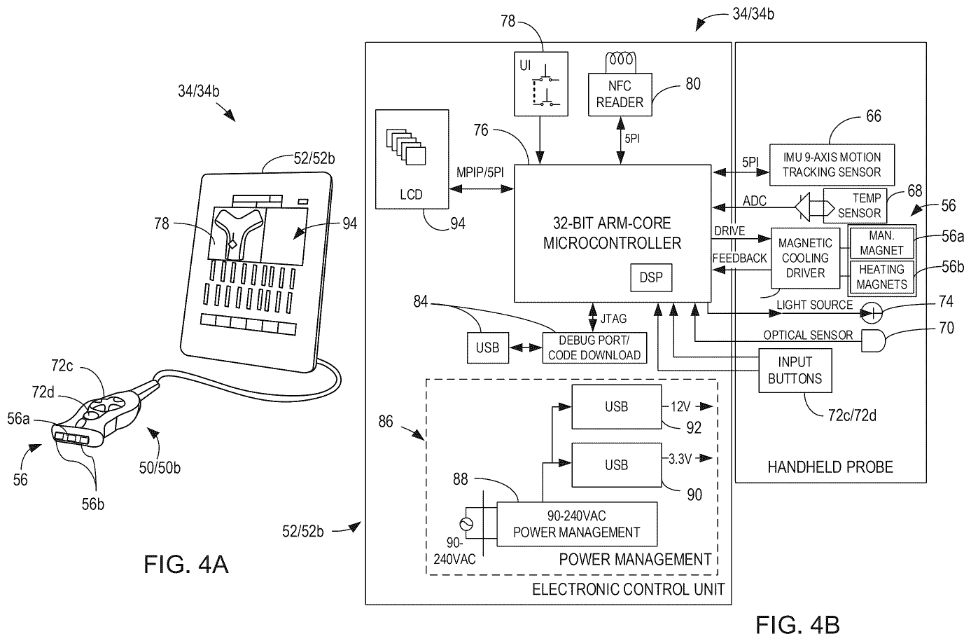

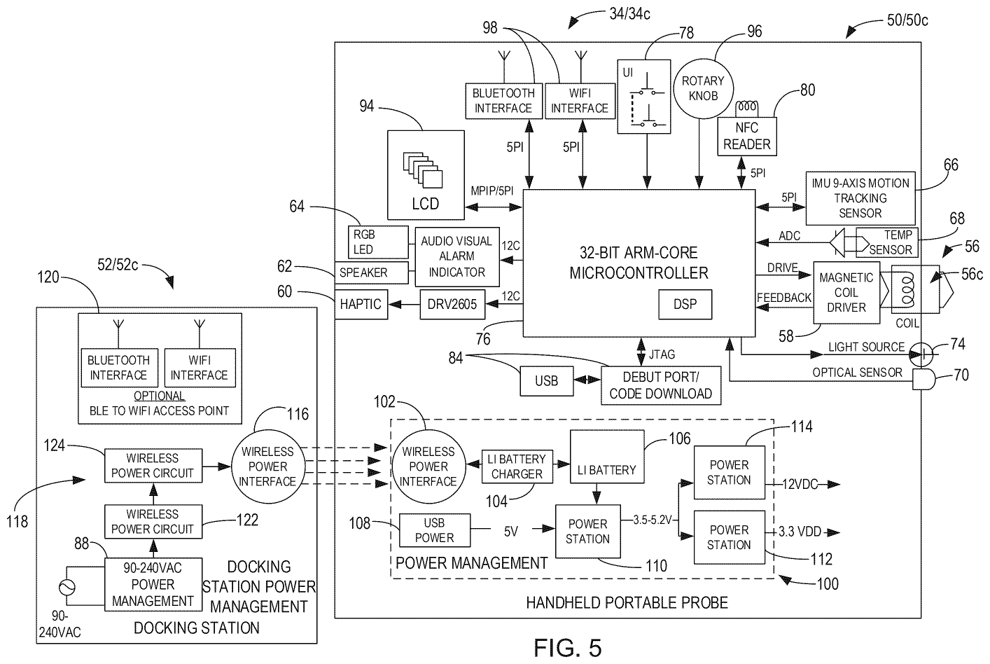

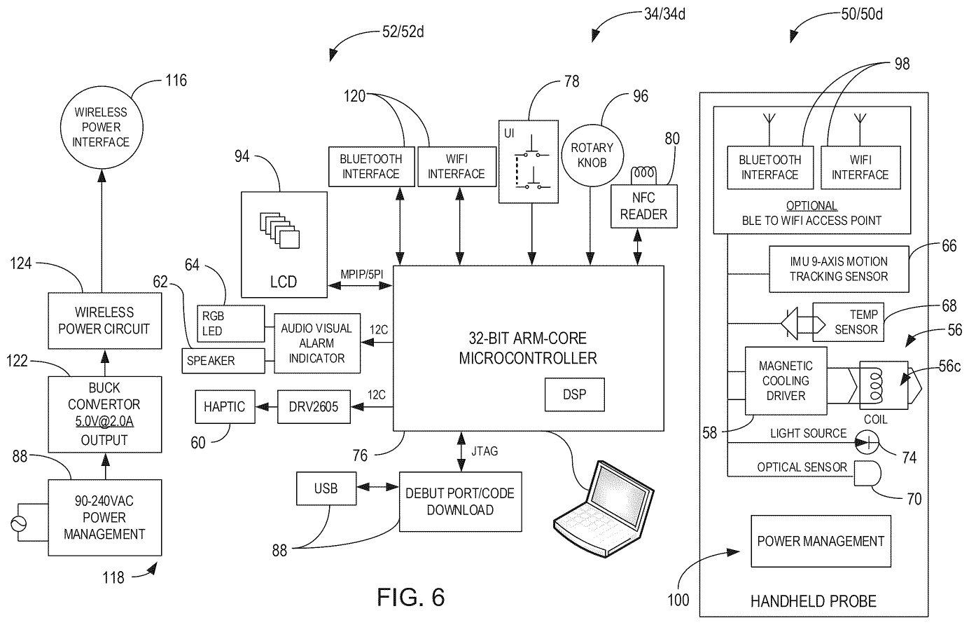

Referring now to FIGS. 3-6, various embodiments of a magnetic probe module 34 are shown, according to aspects of the disclosure. FIGS. 3A and 3B and FIGS. 4A and 4B illustrate magnetic probe modules 34 where the handheld magnetic probe 50 is a wired probe 50a, 50b that is hardwired to the stationary module unit 52, with the stationary module unit 52 provided as an control unit 52a, 52b. FIGS. 5 and 6 illustrate magnetic probe modules 34 where the handheld magnetic probe 50 is provided as a wireless probe 50c, 50d and the stationary module unit 52 comprises a docking station 52c, 52d.

Referring first to FIGS. 3A and 3B, a magnetic probe module 34a is provided according to one embodiment that includes a handheld magnetic probe 50a that is hardwired to a stationary module unit 52 that is in the form of an electronic control unit 52a. A cord 54 connects the magnetic probe 50a with the electronic control unit 52a, with the cord 54 providing power from the electronic control unit 52a to the magnetic probe 50a and transmitting signals between the electronic control unit 52a and the magnetic probe 50a.

As shown in FIG. 3B, the magnetic probe 50a includes a magnet element 56, a driver 58, user feedback elements 60, 62, 64, an array of sensors 66, 68, 70, and input elements 72.

The magnet element 56 may comprise an arrangement of magnets (including a maneuvering magnet 56a and heating magnets 56b) capable of a generating various magnetic fields to selectively manipulate the magnetic nanoparticles contained in the catheter lock solution. The driver 58 is operable to control operation of the magnets 56a, 56b, with the driver 58 receiving a high-power input and controlling the supply of power to the magnets 56a, 56b to control the magnetic field generated thereby. As previously described, the magnets 56a, 56b and driver 58 are operable to cause movement and heating of the magnetic particles in the catheter lock solution—with the probe 50a being operable in a first driving mode that causes the maneuvering magnet 56a to generate a static magnetic field that causes the magnetic nanoparticles to migrate towards the distal end 14 of the catheter 12 and being operable in a second driving mode that causes the heating magnets 56b to generate an alternating magnetic field that causes a temperature of the magnetic nanoparticles to increase, thereby generating a magnetically-induced hyperthermic condition at the distal end 14 of the catheter 12 (i.e., in a distal portion of the standing column of catheter lock fluid contained within the catheter 12) that prevents and/or treats thrombus formation and biofilm accumulation in the catheter 12.

In some embodiments, the user feedback elements 60, 62, 64 of the magnetic probe 50a may comprise a haptic motor 60, an audio alarm 62, and/or a visual indicator 64 that provide an output to a user indicating a state of operation of the magnetic probe 50a. The haptic motor 60 may provide vibrational feedback to a user indicating that an action has responded from a software or hardware command. The audio alarm 62 and/or visual indicator 64 may provide audio and visual feedback indicating that an action has responded from a software or hardware command. As one non-limiting example, the visual indicator 64 may comprise green and red light emitting diodes (LEDs) on the magnetic probe 50a indicating an on/off state of the probe 50a.

In some embodiments, the array of sensors 66, 68, 70 included in the magnetic probe 50a may comprise a motion tracking sensor 66, a temperature sensor 68, and an optical sensor 70 that collectively measure a plurality of parameters during use of the magnetic probe 50a. The motion tracking sensor 66 may, in a non-limiting embodiment, comprise a 9-axis motions sensor that monitors a positioning and orientation of the magnetic probe 50a as it is actuated/navigated by a user. The temperature sensor 68 may monitor a surface/skin temperature of a patient at the site of the indwelling catheter 12, in order to measure/track the in vivo temperature increase that may result from heating of the magnetic nanoparticles by the probe 50a. The optical sensor 70 may operate to visualize/locate the magnetic nanoparticles inside the body of the patient, with the optical sensor 70 operating in connection with an infrared (or near infrared) light source 74 on the magnetic probe 50a that charges the magnetic nanoparticles (i.e., charges the up-conversion nanoparticles attached to the magnetic nanoparticles) and detecting a florescence signal (near infrared signal) of the magnetic nanoparticles after charging thereof, so as to enable location tracking of the magnetic particles within the catheter 12.

The input elements 72 of magnetic probe 50a may comprise buttons that may be depressed by a user to change operational modes of the magnetic probe module 34a. That is, a first button 72a may be pressed by a user to operate the probe 50a in the first driving mode to cause the maneuvering magnet to generate a static magnetic field that causes the magnetic nanoparticles to migrate towards the distal end 14 of the catheter 12, while a second button 72b may be pressed to operate the probe 50a in the second driving mode that causes the heating magnets to generate an alternating magnetic field that causes a temperature of the magnetic nanoparticles to increase.

As further shown in FIG. 3B, the electronic control unit 52a includes a microcontroller (or processor) 76, a user interface 78, a near-field communication (NFC) reader 80, display windows or screens 82, a USB/debug port 84, and a power management arrangement 86.

The power management arrangement 86 may comprise a 90-240VAC medical grade AC to DC stepdown power supply 88 that supplies power to a buck convertor 90 and/or buck/boost convertor 92. The buck convertor 90 operates as a low-level DC power conditioning circuit that provides a low power (e.g., 3.3V) to the electronic control unit 52a and/or magnetic probe 50a, such as to the microcontroller 76 and other peripheral low power devices. The buck convertor 90 operates as a low-level DC power conditioning circuit that provides a regulated power (e.g., 12V) to the electronic control unit 52a and/or magnetic probe 50a, such as to the driver 58.

The USB/debug port 84 provides a port via which service may be performed on the magnetic probe module 34a and/or software updates may be performed on the magnetic probe module 34a.

The NFC reader 80 operates as near field communication tag reader, for reading a patient ID, records, system settings, and the like.

The user interface 78 is configured to enable a user to navigate the control unit 52a and set/adjust settings and parameters (i.e., power output, operational mode, etc.) during operation of the magnetic probe module 34a.

The display windows/screens 82 may display settings to the user.

The microcontroller 76 may comprise a digital signal processor (DSP) and may manage operation of the magnetic probe module 34a. For example, the microcontroller 76 may comprise a communications interface (not shown) for collecting data from sensors and receiving commands from input devices. The microcontroller 76 may also control the driver 58 by use of a digital bus and/or PWM signals, as non-limiting examples.

Referring now to FIGS. 4A and 4B a magnetic probe module 34b is provided according to another embodiment that includes a handheld magnetic probe 50b that is hardwired to a stationary module unit 52 that is in the form of a portable control unit 52b. A cord 54 connects the magnetic probe 50b with the portable control unit 52b, with the cord 54 providing power from the portable control unit 52b to the magnetic probe 50b and transmitting signals between the portable control unit 52b and the magnetic probe 50b.

The magnetic probe 50b may share similar characteristics with the probe 50a of FIGS. 3A and 3B—with the magnetic probe 50b including a magnet element 56, a driver 58, array of sensors 66, 68, 70, and input elements 72.

As shown in FIG. 4A, the magnet element 56 in magnetic probe 50b may differ from that in the probe of FIGS. 3A and 3B but includes a maneuvering magnet 56a and heating magnets 56b that are capable of a generating various magnetic fields to selectively manipulate the magnetic nanoparticles contained in the catheter lock solution. The driver 58 is operable to control operation of the magnets 56a, 56b, with the driver 58 receiving a high-power input and controlling the supply of power to the magnets 56a, 56b to control the magnetic field generated thereby. As previously described, the magnets 56a, 56b and driver 58 are operable to cause movement and heating of the magnetic particles in the catheter lock solution-with the probe 50b being operable in a first driving mode that causes the maneuvering magnet 56a to generate a static magnetic field that causes the magnetic nanoparticles to migrate towards the distal end 14 of the catheter 12 and being operable in a second driving mode that causes the heating magnets 56b to generate an alternating magnetic field that causes a temperature of the magnetic nanoparticles to increase, thereby generating a magnetically-induced hyperthermic condition at the distal end 14 of the catheter 12 (i.e., in a distal portion of the standing column of catheter lock fluid contained within the catheter 12) that prevents and/or treats thrombus formation and biofilm accumulation in the catheter 12.

The array of sensors 66, 68, 70 included in the magnetic probe 50b may be identical to those previously described, and may thus include a motion tracking sensor 66, a temperature sensor 68, and an optical sensor 70 that collectively measure a plurality of parameters during use of the magnetic probe 50b.

The input elements 72 of magnetic probe 50b may comprise a directional control pad 72c and button 72d that may be depressed by a user to change operational modes of the magnetic probe module 34b. That is, the directional control pad 72c and button 72d may be selectively pressed by a user to operate the probe 50b in the first driving mode to cause the maneuvering magnet 56a to generate a static magnetic field that causes the magnetic nanoparticles to migrate towards the distal end 14 of the catheter 12 and operate the probe 50b in the second driving mode that causes the heating magnets 56b to generate an alternating magnetic field that causes a temperature of the magnetic nanoparticles to increase.

The portable control unit 52b may generally function the same as the electronic control unit 52a of FIGS. 3A and 3B but may be provided in a different form factor, with FIG. 4A showing the portable control unit 52b configured as a tablet-style computer or control unit. The portable control unit 52b may thus include a microcontroller (or processor) 76, a user interface 78, a near-field communication (NFC) reader 80, a liquid crystal display (LCD) screen 94, a USB/debug port 84, and a power management arrangement 86.

The LCD screen 94 may operate as a touchscreen that provides a means by which a user can navigate the control unit 52b and set/adjust settings and parameters thereof, with the user interface 78 incorporated with the LCD touchscreen 94. The LCD screen 94 may display settings of the magnetic probe module 34b and/or a value of one or more parameters measured by the magnetic probe 50b. Thus, LCD screen 94 may take the place of feedback elements 60, 62, 64 included in the magnetic probe module 34a.

Referring now to FIG. 5 a magnetic probe module 34c is provided according to another embodiment that includes a wireless magnetic probe 50c and a stationary module unit 52 that is in the form of a docking station 52c.

As shown in FIG. 5, the magnetic probe 50c includes a magnet element 56, a driver 58, user feedback elements 60, 62, 64, an array of sensors 66, 68, 70, a microcontroller (or processor) 76, a user interface 78, a rotary knob 96, a NFC reader 80, a LCD screen 94, a USB/debug port 84, communications interfaces 98, and a power management arrangement 100.

The magnet element 56 may comprise an electromagnetic coil 56c capable of a generating various magnetic fields to selectively manipulate the magnetic nanoparticles contained in the catheter lock solution. The driver 58 is operable to control operation of the electromagnetic coil 56c, with the driver 58 receiving a high-power input and controlling the supply of power to the electromagnetic coil 56c to control the magnetic field generated thereby. As previously described, the electromagnetic coil 56c and driver 58 are operable to cause movement and heating of the magnetic particles in the catheter lock solution-with the probe 50c being operable in a first driving mode that causes the electromagnetic coil 56c to generate a static magnetic field that causes the magnetic nanoparticles to migrate towards the distal end 14 of the catheter 12 and being operable in a second driving mode that causes the electromagnetic coil 56c to generate an alternating magnetic field that causes a temperature of the magnetic nanoparticles to increase, thereby generating a magnetically-induced hyperthermic condition at the distal end 14 of the catheter 12 (i.e., in a distal portion of the standing column of catheter lock fluid contained within the catheter 12) that prevents and/or treats thrombus formation and biofilm accumulation in the catheter 12.

In some embodiments, the user feedback elements 60, 62, 64 of the magnetic probe 50c may comprise a haptic motor 60, an audio alarm 62, and/or a visual indicator 64 that provide an output to a user indicating a state of operation of the magnetic probe 50c. The haptic motor 60 may provide vibrational feedback to a user indicating that an action has responded from a software or hardware command. The audio alarm 62 and/or visual indicator 64 may provide audio and visual feedback indicating that an action has responded from a software or hardware command. As one non-limiting example, green and red light emitting diodes (LEDs) may be provided on the magnetic probe 50c indicating an on/off state of the probe.

In some embodiments, the array of sensors 66, 68, 70 included in the magnetic probe 50c may comprise a motion tracking sensor 66, a temperature sensor 68, and an optical sensor 70 that collectively measure a plurality of parameters during use of the magnetic probe 50c. The motion tracking sensor 66 may, in a non-limiting embodiment, comprise a 9-axis motions sensor that monitors a positioning and orientation of the magnetic probe 50c as it is actuated/navigated by a user. The temperature sensor 68 may monitor a surface/skin temperature of a patient at the site of the indwelling catheter, in order to measure/track the in vivo temperature increase that results from heating of the magnetic nanoparticles by the probe 50c. The optical sensor 70 may operate to visualize/locate the magnetic nanoparticles inside the body of the patient, with the optical sensor 70 operating in connection with an infrared (or near infrared) light source 74 on the magnetic probe 50c that charges the magnetic nanoparticles (i.e., charges the up-conversion nanoparticles attached to the magnetic nanoparticles) and detecting a florescence signal (near infrared signal) of the magnetic nanoparticles after charging thereof, so as to enable location tracking of the magnetic particles within the catheter 12.

The microcontroller 76 may comprise a digital signal processor (DSP) and may manage operation of the magnetic probe 50c. For example, the microcontroller 76 may comprise a communications interface (not shown) for collecting data from sensors 66, 68, 70 and receiving commands from input devices (e.g., user interface 78 and rotary knob 96). The microcontroller 76 may also control the driver 58 by use of a digital bus and/or PWM signals, as non-limiting examples.

The USB/debug port 84 provides a port via which service may be performed on the magnetic probe 50c and/or software updates may be performed on the magnetic probe 50c.

The NFC reader 80 operates as near field communication tag reader 80, for reading a patient ID, records, system settings, and the like.

The LCD screen 94 is configured to display settings of the magnetic probe 50c and/or a value of one or more parameters measured by the magnetic probe 50c.

The user interface 78 is configured to enable a user to navigate operation of the magnetic probe 50c and set/adjust settings and parameters thereof (i.e., operational mode, etc.) during operation of the magnetic probe 50c. In some embodiments, the user interface 78 may comprise physical buttons included on the magnetic probe 50c and/or may be integrated with the LCD screen 94 (i.e., LCD screen 94 may operate as a touchscreen that provides a means by which a user can navigate the magnetic probe 50c and set/adjust settings and parameters thereof, with the user interface 78 incorporated with the LCD touchscreen).

The rotary knob 96 (or directional pad) is provided on the magnetic probe 50c and provides an additional option for navigation and parameter setting/adjustment for the magnetic probe 50c.

The communications interfaces 98 provided on magnetic probe 50c may comprise Wi-Fi and/or Bluetooth interfaces. The communications interfaces 98 enable wireless communication between the magnetic probe 50c and the docking station 52c and/or between the magnetic probe 50c and an external server/network (e.g., a healthcare facility server).

The power management arrangement 100 in magnetic probe 50c may comprise a wireless power interface 102, a battery charger 104, a battery 106, a USB power port 108, a power switch 110, and buck and buck/boost converters 112, 114. The wireless power interface 102 may comprise a power coil that engages/couples with a corresponding wireless power interface 116 on the docking station 52c to receive/harvest power from the docking station 52c and enable charging of the handheld magnetic probe 50c. The battery charger 104 receives power from the wireless power interface 102 for charging of the battery 106 included in magnetic probe 50c (e.g., a lithium-ion battery). In some embodiments, a USB power port 108 provides an additional means for charging the battery 106, with a power switch 110 provided to allow for charging of the battery 106 via either of the USB power port 108 or the wireless power interface 102. Power may be provided from the battery 106 to the buck convertor 112 and/or buck/boost convertor 114. The buck convertor 112 operates as a low-level DC power conditioning circuit that provides a low power (e.g., 3.3V) to components of the magnetic probe 50c, such as to the microcontroller 76 and other peripheral low power devices. The buck convertor 114 operates as a low-level DC power conditioning circuit that provides a regulated power (e.g., 12V) to components of the magnetic probe 50c, such as to the driver 58.

As further shown in FIG. 5, the docking station 52c comprises a power management arrangement 118 that provides for charging of the magnetic probe 50c and a communications interface 120. The power management arrangement 118 may comprise a 90-240VAC medical grade AC to DC stepdown power supply 88 that supplies power to a buck convertor 122 that performs a low-level DC power conditioning (e.g., provides a 5.0V@2.0 Amp output). The buck convertor 122 may provide an output to a wireless power management circuit 124 and wireless power interface (e.g., wireless power coil) 116 configured to transmit the power to the handheld magnetic probe 50c when engaged with the docking station 52c.

The communications interface 120 of docking station 52c may comprise a Wi-Fi and/or Bluetooth interface that provides a bridge or access point to convert/transmit data from the handled magnetic probe 50c to an external system or database, such as a cloud network of a healthcare facility.

Referring now to FIG. 6, a magnetic probe module 34d is provided according to another embodiment that includes a wireless magnetic probe 50d and a stationary module unit 52 that is in the form of a docking station 52d. Wireless communication and charging between the magnetic probe 50d and docking station 52d is similar to as described in the module 34c of FIG. 5, but the magnetic probe 50d and docking station 52d are modified in the module 34d of FIG. 6—with the magnetic probe 50d configured as a more “simplified” probe and the docking station 52d including many of the processing, display, and alert features previously incorporated into the magnetic probe 50d in FIG. 5.

As shown in FIG. 6, the magnetic probe 50d includes a magnet element 56, a driver 58, an array of sensors 66, 68, 70, communications interfaces 98, and a power management arrangement 100. Each of these components may be similar in structure and operation to the corresponding components in the magnetic probe 50c of FIG. 5, and thus are not described in detail here.

As also shown in FIG. 6, the docking station 52d comprises a microcontroller (or processor) 76, user feedback elements 60, 62, 64, a user interface 78, a rotary knob 96, an NFC reader 80, an LCD screen 94, a USB/debug port 84, communications interfaces 120, and a power management arrangement 118. Again, each of these components may be similar in structure and operation to the corresponding components in the magnetic probe 50c and docking station 52c of FIG. 5, and thus are not described in detail here.

With the magnetic probe module 34d shown in FIG. 6, all data processing, parameter setting, parameter display, and notification generation/output is performed at the docking station 52d, with the magnetic probe 50d thus being configured as a probe 50d with less or no features thereon via which a user sets or modifies operational parameters for the probe 50d.

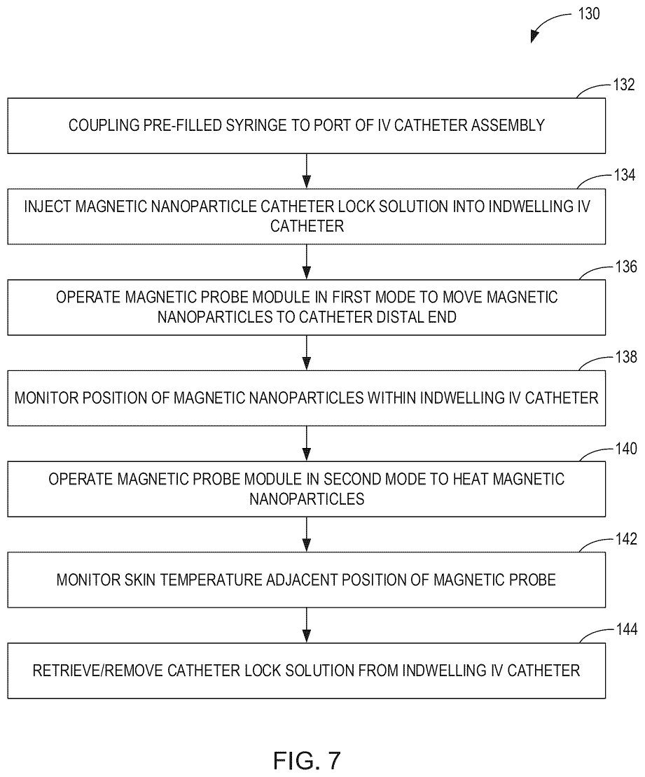

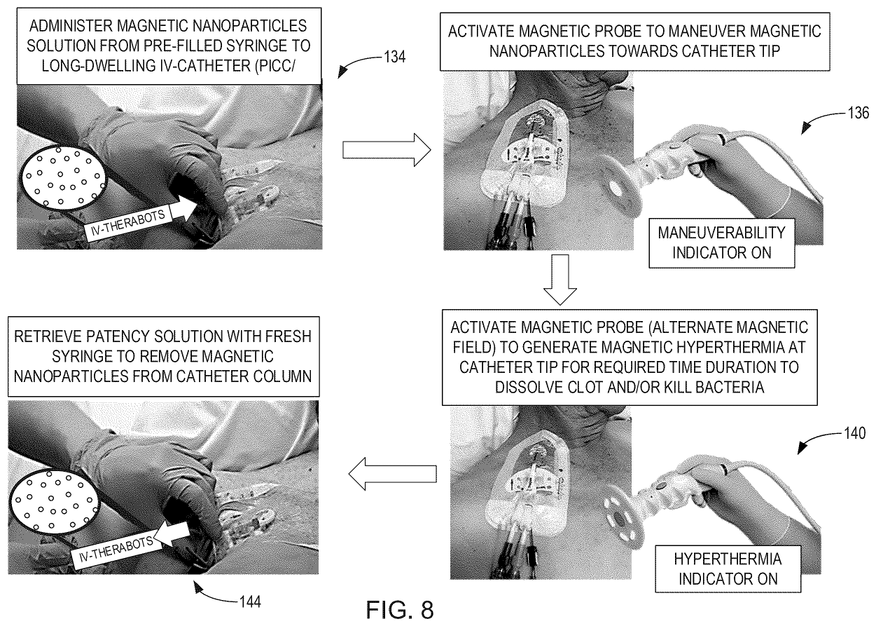

Referring now to FIGS. 7-9, a flowchart and accompanying graphical views are provided for a method 130 for preventing and/or treating thrombus formation and biofilm accumulation in an indwelling IV catheter, according to an aspect of the disclosure. The method 130 may be performed by the system 30 of FIG. 2, including use of any of the magnetic probe modules 34 shown and described in FIGS. 3-6.

The method 130 begins at step 132 by coupling a pre-filled syringe 32 to a port 22 of an IV catheter assembly 10 having a catheter 12 with a distal end 14 positioned intravenously within a patient, with the port 22 providing fluid access to the catheter 12.

Upon coupling of the pre-filled syringe 32 to the port, the method 130 continues at step 134 with the injecting of an aqueous catheter lock solution into the catheter 12 via the pre-filled syringe 32. As previously described, the aqueous catheter lock solution includes magnetic particles suspended therein that may comprise magnetic nanoparticles composed of iron oxide or iron-oxides phases (i.e., Fe₃O₄ and Fe₂O₃) configured as nanorods. In some embodiments, other magnetic ferrites in spinel structures doped with Mn, Co, Ni or Zn may also form part of the magnetic nanoparticles. Additionally, the magnetic nanoparticles in the catheter lock solution may be loaded with an antithrombotic and/or antimicrobial material that enables prevention and/or treatment of thrombotic occlusions and/or biofilms and microbes that may be present at the distal end/tip 14 of the catheter 12. Still further, the magnetic nanoparticles may be coated with up-conversion nanoparticles that shift a florescence signal of the magnetic nanoparticles into near infrared when the up-conversion nanoparticles are charged by an infrared or near infrared light source 74.

With the catheter lock solution injected into the indwelling catheter 12, a magnetic probe module 34 may then be operated to induce/generate a magnetically-induced hyperthermic condition at the distal end 14 of the catheter 12, in order to prevent and/or treat thrombus formation and biofilm accumulation thereat.

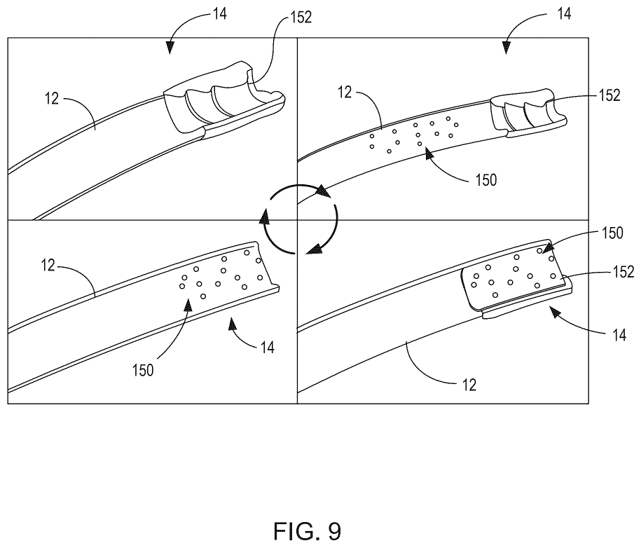

At step 136 of the method 130, the magnetic probe module 34 is operated in a first driving mode that causes the magnetic nanoparticles to move towards the distal end 14 of the catheter 12. A magnet element 56 (which may comprise a magnetic coil or other magnet arrangement) in the magnetic probe 50 is driven by a corresponding magnetic driver 58 such that the magnet element 56 generates a static magnetic field that causes the magnetic nanoparticles to migrate towards the distal end 14 of the catheter 12. The movement of the magnetic nanoparticles towards the distal end 14 of the catheter 12 is illustrated in FIG. 9—with magnetic nanoparticles 150 shown as moving toward the distal end 14. As a non-limiting example, a static magnetic field may be generated at 30-70 Gauss and 5-50Hz for a period of 10-20 minutes, in order to cause the magnetic nanoparticles to migrate to the distal end 14 of the catheter 12.

In one embodiment, as the magnetic probe module 34 is operating in the first driving mode, a monitoring/tracking of the position of the magnetic nanoparticles within the catheter 12 may be performed at step 138, in order to determine if the magnetic nanoparticles have been moved sufficiently toward the distal end 14 of the catheter 12. As previously described, the magnetic particles may be coated with up-conversion nanoparticles that shift a florescence signal of the magnetic particles into near infrared. Thus, at step 138, the method 130 may include emitting infrared or near infrared light from a light source 74 toward the magnetic nanoparticles that are coated with the up-conversion nanoparticles (in order to charge the up-conversion nanoparticles) and then subsequently detecting the florescence signal of the magnetic particles via an optical sensor 70, thereby enabling location tracking of the magnetic nanoparticles within the catheter 12.

At step 140 of the method 130, and upon the magnetic nanoparticles being moved to the distal end 14 of the catheter 12, the magnetic probe module 34 is operated in a second driving mode that causes a temperature of the magnetic particles to increase. A magnet element 56 (which may comprise a magnetic coil or other magnet arrangement) in the magnetic probe 50 is driven by a corresponding magnetic driver 58 such that the magnet element 56 generates an alternating magnetic field that heats the magnetic nanoparticles. That is, the alternating magnetic field activates the magnetic nanoparticles via magnetic coupling between a magnetic component of the magnetic field and a magnetic moment of the magnetic nanoparticles, with the magnetic nanoparticles absorbing the energy from this coupling and dissipating it as heat.

According to aspects of the disclosure, operating the magnetic probe 50 in the second driving mode at step 140 may comprise pre-programming the magnetic probe 50 to run for a pre-set period of time required to achieve a target temperature for the aqueous catheter lock solution that creates the magnetically-induced hyperthermic condition at the distal end 14 of the catheter 12. In some embodiments, design optimization studies may be used to pre-calibrate operation of the magnetic probe module 34, with an algorithm then being provide to the magnetic probe module 34 (i.e., the microcontroller 76 thereof) defining the input signal required to achieve a target temperature that will be sufficient to kill microbes (treat a biofilm) and dissolve clots at the distal end 14 of the catheter 12, including how long heating of the magnetic nanoparticles must be performed in the second driving mode. FIG. 9 illustrates heated magnetic nanoparticles 150 creating a magnetically-induced hyperthermic condition at the distal end 14 of the catheter 12 in order to remove a biofilm and dissolve a clot (collectively indicated at 152).

As a non-limiting example, the magnetic probe module 34 may be operated in the second driving mode at 300-600 Gauss and 100-500 Hz for a period of 10-20 minutes, in order to raise the temperature within the lumen of the catheter 12 to a temperature of 40-70 degrees Celsius and generate a hyperthermic condition sufficient to prevent and/or treat thrombus formation and biofilm accumulation thereat.

During operation of the magnetic probe module 34 in the second driving mode, other steps may be performed in the method 130 in order to monitor certain conditions associated with heating of the magnetic nanoparticles. In one embodiment, as the magnetic probe module 34 is operating in the second driving mode, a temperature at the location at which the magnetic probe 50 is positioned adjacent the skin is monitoring at step 142. As previously described, a temperature sensor 68 included in the magnetic probe 50 may function to measure a skin temperature of the patient at this location. In some embodiments, monitoring of the temperature in this fashion may aid in determining when operation of the magnetic probe module 34 in the second driving mode should be stopped.

Upon generating the magnetically-induced hyperthermic condition at the distal end 14 of the catheter 12-for a sufficient period of time and at a sufficient temperature, which may be determined according to pre-set parameters-the method 130 may continue at step 144 with retrieval of the aqueous catheter lock solution out from the catheter 12. In some embodiments, an empty syringe 128 may be connected to a port 22 of the catheter assembly 10, with the syringe 128 actuated to aspirate the catheter lock solution out from the catheter 12 and into the syringe 128.

It is recognized that a decision to perform the method 130 may be made in various ways or for various reasons. As one example, it may be decided to perform the method when a user experiences difficulty/resistance while aspirating locking solution before medication administration, as this could be an indication of catheter occlusion and a cue to initiate maneuvering and hyperthermia to prevent and/or treat a clot or biofilm. As another example, it may be decided to perform the method as a prophylactic measure, with maneuvering and hyperthermia episodes being run at pre-determined frequencies during catheter dwell time and in-between medication waiting periods to reduce risk of clot/biofilm formation.

Beneficially, embodiments of the disclosure described herein provide systems and methods for maintaining patency of an indwelling IV catheter, such as a peripherally inserted central catheter (PICC) or central venous catheter (CVC) as non-limiting examples, with the systems/methods operating to introduce a catheter lock solution having magnetic particles suspended therein into the IV catheter and generate a hyperthermic condition at the distal end of the IV catheter via the catheter lock solution, so as to prevent and/or treat blood clots/biofilms within the catheter.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

1. A system comprising:
an intravenous (IV) catheter assembly including a catheter having a distal end positionable intravenously within a patient and a port providing fluid access to the catheter, wherein a lumen of the catheter may retain a fluid therein; and
a pre-filled syringe connectable to the port, the pre-filled syringe comprising a syringe barrel and a plunger assembly movable within the syringe barrel, with the syringe barrel defining a chamber that contains an aqueous catheter lock solution having magnetic particles suspended therein; and
a probe module comprising a magnetic probe that includes a magnet element and a driver, the magnetic probe positionable adjacent the IV catheter assembly and to cause movement and heating of the magnetic particles in the aqueous catheter lock solution;
wherein the magnetic probe is configured to:
operate in a first driving mode where the magnet element causes the magnetic nanoparticles to migrate towards the distal end of the catheter; and
operate in a second driving mode where the magnet element causes a temperature of the magnetic nanoparticles to increase, generating a magnetically-induced hyperthermic condition at the distal end of the catheter;
wherein the magnetically-induced hyperthermic condition at the distal end of the catheter prevents and/or treats thrombus formation and biofilm accumulation in the catheter.

2. The system of claim 1, wherein the magnetic particles comprise iron oxide nanoparticles or similar magneto-responsive material particles.

3. The system of claim 2, wherein the magnetic particles are loaded with an antithrombotic or antimicrobial material.

4. The system of claim 1, wherein the magnet element comprises a magnetic coil or a plurality of magnets.

5. The system of claim 1, wherein when operating in the second driving mode, the magnetic probe is configured to generate an alternating magnetic field that causes a temperature of the magnetic particles to increase.

6. The system of claim 5, wherein the alternating magnetic field activates the magnetic particles via magnetic coupling between a magnetic component of the magnetic field and a magnetic moment of the magnetic particles, with the magnetic particles absorbing the energy from this coupling and dissipating it as heat, to generate the hyperthermic condition.

7. The system of claim 6, wherein when operating in the second driving mode, the magnetic probe is configured to heat the aqueous catheter lock solution to 40-70 degrees Celsius.

8. The system of claim 1, wherein the magnetic probe comprises a temperature sensor to measure skin temperature at a location adjacent the distal end of the catheter.

9. The system of claim 1, wherein the magnetic particles are coated with up-conversion nanoparticles that shift a florescence signal of the magnetic particles into near infrared.

10. The system of claim 9, wherein the magnetic probe further comprises:
an infrared or near infrared light source configured to charge the up-conversion nanoparticles; and
an optical sensor configured to detect the florescence signal of the magnetic particles after charging thereof, so as to enable location tracking of the magnetic particles within the catheter.

11. The system of claim 1, wherein the probe module further comprises:
a display configured to display settings of the magnetic probe and/or a value of one or more parameters measured by the magnetic probe; and
a user interface configured to input settings for the magnetic probe;
wherein the display and the user interface are provided on the probe or on a docking station or electronic control unit associated with the magnetic probe.

12. The system of claim 1, wherein the magnetic probe has an indicator light configured to indicate whether the magnetic probe is operating in the first driving mode or the second driving mode.

13. A method for preventing and/or treating thrombus formation and biofilm accumulation in an indwelling IV catheter, the method comprising:
coupling a pre-filled syringe to a port of an intravenous (IV) catheter assembly including a catheter having a distal end positioned intravenously within a patient, wherein the port provides fluid access to the catheter;
injecting an aqueous catheter lock solution into the catheter via the pre-filled syringe, the aqueous catheter lock solution having magnetic particles suspended therein;
operating a magnetic probe to cause movement and heating of the magnetic particles in the aqueous catheter lock solution injected into the catheter, wherein operating the magnetic probe comprises:
operating the magnetic probe in a first driving mode to cause the magnetic nanoparticles to migrate towards the distal end of the catheter; and
operating the magnetic probe in a second driving mode to cause a temperature of the magnetic nanoparticles to increase, generating a magnetically-induced hyperthermic condition at the distal end of the catheter;
wherein the magnetically-induced hyperthermic condition at the distal end of the catheter prevent and/or treats thrombus formation and biofilm accumulation in the catheter.

14. The method of claim 13, wherein operating the magnetic probe in the second driving mode comprises generate an alternating magnetic field that causes a temperature of the magnetic particles to increase.

15. The method of claim 13, further comprising measuring skin temperature at a location adjacent the distal end of the catheter magnetic probe via a temperature sensor included in the magnetic probe.

16. The method of claim 13, wherein the magnetic particles are coated with up-conversion nanoparticles that shift a florescence signal of the magnetic particles into near infrared, and wherein the method further comprises:
emitting infrared or near infrared light from a light source toward the magnetic particles, in order to charge the up-conversion nanoparticles; and
detecting the florescence signal of the magnetic particles after charging thereof via an optical sensor, so as to enable location tracking of the magnetic particles within the catheter.

17. The method of claim 13, wherein operating the magnetic probe in the second driving mode comprises pre-programming the magnetic probe to run for a pre-set period of time required to achieve a target temperature for the aqueous catheter lock solution that creates the magnetically-induced hyperthermic condition at the distal end of the catheter.

18. The method of claim 17, wherein the target temperature of the aqueous catheter lock solution is 40-70 degrees Celsius.

19. The method of claim 13, wherein the magnetic particles are loaded with an antithrombotic or antimicrobial material.

20. The method of claim 13, further comprising retrieving the aqueous catheter lock solution out from the catheter after generating the magnetically-induced hyperthermic condition at the distal end of the catheter.

21. A system comprising:
a tubular structure having a distal end positionable intravenously within a patient and a lumen;
a pre-filled syringe that may be placed in fluid communication with the tubular structure, the pre-filled syringe comprising a syringe barrel and a plunger assembly movable within the syringe barrel, with the syringe barrel defining a chamber that contains an aqueous catheter lock solution having magnetic particles suspended therein; and
a probe module comprising a magnetic probe that includes a magnet element and a driver, the magnetic probe positionable in proximity to the tubular structure to cause movement and heating of the magnetic particles in the aqueous catheter lock solution;
wherein the magnetic probe is configured to:
operate in a first driving mode where the magnet element causes the magnetic nanoparticles to migrate towards the distal end of the tubular structure; and
operate in a second driving mode where the magnet element causes a temperature of the magnetic nanoparticles to increase, generating a magnetically-induced hyperthermic condition at the distal end of the tubular structure;
wherein the magnetically-induced hyperthermic condition at the distal end of the tubular structure prevents and/or treats thrombus formation and biofilm accumulation in the tubular structure.
